(19) United States
(12) Patent Application Publication
(10) Pub. No.: US 2006/0281967 A1
(43) Pub. Date: Dec. 14, 2006

(54) Prosthetic safeguard for support implants

(75) Inventors: Alfredo Meneghin, Anse (FR); Michel Therin, Lyon (FR)

Correspondence Address: OLIFF & BERRIDGE, PLC, P.O. BOX 19928, ALEXANDRIA, VA, 22320, US

(73) Assignee: SOFRADIM PRODUCTION, TREVOUX (FR)

(21) Appl. No.: 11/409,025

(22) Filed: Apr. 24, 2006

Related U.S. Application Data
(60) Provisional application No. 60/673,767, filed on Apr. 22, 2005.

(51) Int. Cl. A61F 2/02; A61F 2/00

(52) U.S. Cl. 600/37; 623/23.74

(57) ABSTRACT

The present invention relates to a prosthetic openwork knit for the treatment of urinary incontinence and/or prolapse, based on an arrangement of yarns of a biocompatible polymer comprising at least one first sheet defining a first chain structure, in which knit said arrangement of yams further comprises at least two non-meshing sheets, of partial weft, the number of chain yarns in said chain structure being from 6 to 12. The invention also relates to a support implant for the treatment of stress urinary incontinence and/or prolapse obtained from this knit, and to the method of producing such a knit.

The present invention relates to a prosthetic openwork knit for the treatment of urinary incontinence and/or prolapse, particularly for producing bands or tapes for urethral support in the treatment of female stress incontinence, and the treatment of what are generally mainly female pelvic floor disorders, also known as prolapse.

### BACKGROUND OF THE INVENTION

The surgical treatment of female stress incontinence usually involves the use of reinforcements in the form of tapes placed underneath the middle urethra. The central part of the implant is placed below this organ and may be in contact with it in order to support it while the lateral parts of the implant are attached to stable anatomic parts such as the abdominal wall, the posterior face of the OS pubis, or the obturator membrane, for example by means of staples, sutures or simple tissue anchoring.

As far as the treatment of prolapse is concerned, part of the implant is positioned against or near the organ to be supported and part against stable anatomic parts such as the abdominal wall, the posterior face of the OS pubis, the obturator membrane, the promontory of the sacrum, or the sacrosciatic ligaments, for example by means of staples, sutures or simple tissue anchoring.

As is known, such a support implant must satisfy many demands, and in particular must have appropriate mechanical strength, particularly in the longitudinal direction, and be biocompatible and flexible. These support implants must also be macroporous so as to integrate intimately and quickly into the receiver's tissues without interfering with the hollow viscera with which they are in contact when implanted. These implants are advantageously made from biocompatible monofilament in order to develop the least possible surface area that could encourage bacterial colonization. These support implants may be suturable. The implants may also advantageously be relatively inextensible longitudinally so that they can easily be pulled along sometimes tortuous anatomical paths. Lastly, it is desirable that these support implants be adapted to the anatomy and morphology of the patient in both breadth and length.

### DESCRIPTION OF THE PRIOR ART

One essential property of these implants is their mechanical strength, which must be very great in order to support the organs to be treated. To increase this strength, it has been proposed that the amount of material used in the implants be increased, as by using thicker and therefore stronger yarns, or by making a denser lattice.

However, since such implants are designed to be left permanently in the patient's body, it is undesirable to increase the amount of material used in these treatments, such being contrary to present-day criteria of tolerance and of tissue integration of support and reinforcement implants.

Another problem that occurs with support tapes is their curling. For the purposes of the present application, “curling” means the spontaneous rolling up of the tape upon itself, about its longitudinal axis, when stretched in the direction of its length. In this form, these implants must maintain adequate mechanical (particularly strength) properties while minimizing the release of particles, that is ends of yarns when under stress and must allow mechanically stable tissue anchoring.

There is therefore a need for a knit, especially one that is macroporous and made from monofilament, that can be used to produce support implants, particularly in the form of tapes, having both excellent mechanical strength and the least possible mass per unit area.

It is an object of the present invention to fulfill this need by providing a knit having a particular arrangement of yarns, in particular having at least one meshing sheet and at least two non-meshing sheets, making it possible to produce support tapes that have great mechanical strength and are very lightweight and stable.

### SUMMARY OF THE INVENTION

The present invention relates to a prosthetic openwork knit for the treatment of urinary incontinence and/or prolapse, based on an arrangement of yarns of a biocompatible polymer comprising at least one first sheet defining a first chain structure, in which knit said arrangement of yarns further comprises at least two non-meshing sheets, of partial weft, the number of chain yarns in said chain structure being from 6 to 12.

The present invention also relates to the use of a prosthetic knit as above to obtain a prosthetic product for surgical use, particularly to obtain a support implant for the treatment of stress urinary incontinence and/or prolapse.

The knit according to the invention can be used directly as a support implant for the treatment of stress urinary incontinence and/or prolapse or may be cut up transversely to obtain such an implant.

The invention also relates to a support implant for the treatment of stress urinary incontinence and/or prolapse, which is obtained by cutting transversely a prosthetic knit as above.

The present invention also relates to a method of producing a prosthetic openwork knit as above that comprises the following steps:
a) a knitted structure is produced on a warp or Raschel machine as a first sheet threaded continuously or as needed and obtained from a first guide bar, the chart followed for the knitting of the yarns of said first sheet leading to the formation of a chain, and at least a first non-meshing sheet and a second non-meshing sheet, said non-meshing sheets being threaded continuously or as needed, each said non-meshing sheet being obtained from a guide bar, the chart followed for the knitting of the yarns of each non-meshing sheet being such that for every n yarns (A) of the chain structure, n ranging from 6 to 12, every yarn of said non-meshing sheets that approaches chain yarn n+1 (A1, A11, A12) turns back one hundred and eighty degrees at said chain yarn n+1, said chain yarn n+1 being termed the free chain yarn, and
b) said free chain yarns are unroved along the length of the knitted structure obtained in step a) and said free chain yarns are removed to produce knits in which the number of chain yarns is from 6 to 12.

The knit or implant according to the invention has excellent mechanical strength, in particular an excellent tensile strength, can be relatively inelastic, and is therefore ideal for producing a support implant for the treatment of stress urinary incontinence and prolapse, without the use of a protective sheath being necessary.

Moreover, because of the specific arrangement of yarns which it comprises, particularly due to the presence of two non-meshing sheets, the knit or implant according to the invention is very light, yet has sufficient mechanical strength to support the organs to be treated. Thus, the knit or implant according to the invention includes a minimal amount of yarn and therefore of material but is nevertheless strong enough to support the organs to be treated.

In particular, the presence of two non-meshing sheets, generally two intersecting non-meshing sheets, i.e. their respective guide bars move symmetrically with respect to each other and are offset one with respect to the other in the direction of production of the knitted structure on the knitting machine, makes it possible to obtain knits and/or tapes and/or implants that have good resistance to lateral compression. Consequently, when the two opposite longitudinal edges of a knit, tape and/or implant according to the invention, obtained from said knitted structure, are compressed, for example between two fingers, this knit, tape and/or implant retains roughly the same width. The loss of width of a knit, tape and/or implant according to the invention when its two opposite longitudinal edges are squeezed between two fingers is preferably less than 10%. The knit, tape and/or implant according to the invention therefore has great stability during manipulation, passage through any auxiliary equipment (the eye of a needle, a canula or the like) and in the tissues of the patient (limiting the string effect).

Owing to its particular arrangement of yarns, there is therefore no risk of unroving of the knit, tape and/or implant according to the invention.

Again, owing to its method of production, this knit has edges that are atraumatic and stable, meaning that it will not fray or release particles and can therefore be introduced into the tissues without a protective sheath. In addition, all the knits, tapes or implants obtained from one knitted structure have uniform heat-setting and are easy to handle.

In the present application the expression “prosthetic knit” means a knit designed to be implanted into a human or animal in the form of a prosthesis or in the form of any other articles fashioned at least partly with said knit.

In the present application the expression “openwork knit” means a knit whose structure or structures create cells or holes through the thickness of the knit, and these cells or holes can act as channels leading from one side of the knit to the other. Such an openwork (or “macroporous”) knit will integrate better into the tissues.

The expression “meshing sheet” means, in the present application, a sheet of yarns in which the chart followed for the knitting of the yarns leads to the formation of meshes. As is known, a chain-structured sheet is a meshing sheet, whereas sheets with partial weft are non-meshing sheets.

In the present application the expression “free chain yarn” means a chain yarn with no weft yarn completely passing through it, in other words a chain yarn in which, all the way along the longitudinal dimension of the knitted structure, any weft yarn approaching and interacting with this chain yarn, for example by being linked to it, then turns back one hundred and eighty degrees on reaching this chain yarn.

In the present application,
the mass per unit area of a knit is measured in accordance with standard ISO 3801,
the tensile strength of a knit in the longitudinal direction and in the transverse direction is measured in accordance with standard ISO 13934-1, and
the elongation under 2 daN in the longitudinal dimension is measured in accordance with standard ISO 13934-1.

Preferably the number of chain yarns in the knit according to the invention is from 8 to 11, and is preferably 9. With about this number of chain yarns it is possible to obtain a knit that has good mechanical strength in the length direction and good longitudinal and transverse stability.

The knit is preferably based on monofilament or multifilament yarns of a biocompatible polymer material selected from polypropylene, polyester, polyamide and blends thereof. Said biocompatible polymer is advantageously polypropylene.

In another embodiment, the knit according to the invention is based on monofilament or multifilament yarns of a biocompatible and bioresorbable polymer.

In yet another embodiment, the knit according to the invention can be made from a blend of bioresorbable biocompatible yarns and non-bioresorbable biocompatible yarns. It is thus possible to make temporarily reinforced implants whose skeleton must remain permanently inside the patient's body for a permanent minimal support.

The knit according to the invention is preferably based on monofilament yarns having a diameter of from 0.05 mm to 0.15 mm, preferably approximately 0.10 mm.

With such a diameter, associated with the particular arrangement of yarns of the knit according to the invention, it is possible to achieve excellent mechanical strength without having to add to the amount of material by using thick yarns.

The knit according to the invention preferably has a thickness of from 0.20 mm to 0.40 mm, preferably approximately 0.30 mm.

In a preferred embodiment of the invention, the knit comprises cells having a diameter of from 0.3 to 1.5 mm, preferably of from 0.3 to 0.9 mm. With such a structure there is improved tissue anchoring.

The knit according to the invention preferably has a width of from 0.6 cm to 1.5 cm.

The knit according to the invention preferably has a mass per unit area which is from 40 to 75 g/m², and is preferably from 50 to 60 g/m².

Advantageously, the tensile strength of the knit according to the invention in the longitudinal and transverse directions, measured according to standard ISO 13934-1 is from 20 to 90 N, preferably from 40 to 90 N, preferably from 55 to 75 N, and more preferably from 60 to 70 N.

The knit or implant according to the invention therefore combines excellent mechanical strength, or tensile strength, with a small mass per unit area, while being relatively inelastic and insensitive to the modifications associated with the conditions of use such as curling, stringing or deformations in the transverse direction, and release of particles. An implant of this kind is advantageous because it can be used to give efficient support to the organs to be treated while minimizing the mass of the implanted foreign body.

The knit according to the invention preferably has an extension under 2 daN in the longitudinal direction, measured according to standard ISO 13934-1, of less than or equal to 15%, more preferably less than or equal to 10%.

The knit according to the invention preferably comprises a first non-meshing sheet and a second non-meshing sheet, said first non-meshing sheet being in accordance with the chart 1-1/3-3/2-2/0-0//, said second non-meshing sheet being in accordance with the chart 3-3/2-2/0-0/1-1//.

Such charts are highly advantageous because they keep the chain yarns better in position, have excellent transverse and longitudinal mechanical strength, and keep the cells as large as possible, without adding material.

In an embodiment of the invention, the knit according to the invention has a number of stitch courses per centimeter ranging from 13 to 18. Preferably, this number of stitch courses per centimeter is 15. Such a number of stitch courses per centimeter allows a better holding and a better fixing of the knitted structure. The knit according to the invention is not loose and is quite dense.

The knit according to the invention is preferably heat-set.

In one embodiment of the invention, the knit according to the invention has a length of from 10 to 50 cm and constitutes a support implant for the treatment of stress urinary incontinence and/or prolapse.

In another embodiment of the invention, an implant according to the invention is made by cutting the knit according to the invention in the transverse direction. The implant according to the invention preferably has a length of from 10 to 50 cm.

The knit according to the invention is preferably produced by a method comprising the following steps:
a) a knitted structure is produced on a warp or Raschel machine as a first sheet threaded continuously or as needed and obtained from a first guide bar, the chart followed for the knitting of the yarns of said first sheet leading to the formation of a chain, and at least a first non-meshing sheet and a second non-meshing sheet, said non-meshing sheets being threaded continuously or as needed, each said non-meshing sheet being obtained from a guide bar, the chart followed for the knitting of the yarns of each non-meshing sheet being such that for every n yarns (A) of the chain structure, n of from 6 to 12, every yarn of said non-meshing sheets that approaches chain yarn n+1 (A1, A11, A12) turns back one hundred and eighty degrees at said chain yarn n+1, said chain yarn n+1 being termed the free chain yarn, and
b) said free chain yarns are unroved along the length of the knitted structure obtained in step a) and said free chain yarns are removed to produce knits in which the number of chain yarns is from 6 to 12.

Therefore, according to the method of manufacturing the knit according to the invention, all the chain yarns, be they free or not, are knitted with the same guide bar.

The value n is preferably from 8 to 11 and, more preferably, n is 9.

In a preferred embodiment of the method according to the invention, the yarns of the first chain-structured sheet are knitted in accordance with a chart 1-0/0-1//, the yarns of the first non-meshing sheet are knitted in accordance with a chart 1-1/3-3/2-2/0-0//, and the yarns of the second non-meshing sheet are knitted in accordance with a chart 3-3/2-2/0-0/1-1//.

Preferably, the guide bar of the first chain-structured sheet is continuously full-threaded, the guide bar of the first non-meshing sheet is threaded continuously 1 full, 1 empty, 3 full, 1 empty, 1 full, 3 empty, and the guide bar of the second non-meshing sheet is threaded continuously 1 full, 1 empty.

The two guide bars of the two non-meshing sheets advantageously move in partial weft under three needles, symmetrically with respect to each other, each offset with respect to the other in the direction of production of the knitted structure. Such a knitting pattern, with the weft bars moving symmetrically with respect to each other and therefore intersecting each other, holds the chains more securely and therefore has better resistance to lateral compression of the knits, implants and/or tapes according to the invention obtained from this knitted structure.

The knitted structure preferably undergoes heat-setting between step a) and step b). The knitted structure is thus easy to manipulate, particularly in the unroving step. Also, all knits and implants according to the invention obtained from any one heat-set knitted structure have uniform heat-setting, which ensures better uniformity of the physical and mechanical properties from one tape to the next following unroving.

### BRIEF DESCRIPTION OF THE DRAWINGS

A clearer understanding of the invention will be gained from the description which follows with reference to the accompanying drawings:

FIG. 1 is a simplified diagram of a knitted structure comprising a first sheet of chain structure and two non-meshing sheets from which the knit according to the invention can be obtained;

FIG. 2 shows a knitted structure from which the knits according to the invention can be obtained, from which two free chain yarns have been partially unroved;

FIG. 3 is the drawing of a view under an Itashi S 800 scanning electron microscope, enlarged 20×, of the unroving of a free chain yarn from a knitted structure, enabling knits according to the invention to be obtained;

FIG. 4 is the drawing of a view under an Itashi S 800 scanning electron microscope, enlarged 20×, of the center of a knit or implant according to the invention.

### DESCRIPTION OF THE PREFERRED EMBODIMENTS

In FIG. 1, E-E′ shows, for a knitted structure from which a knit according to the invention can be obtained, the transverse direction or dimension of the knit; F-F′ the longitudinal direction or dimension of the knit; and G-G′ the diagonal direction or dimension of the knit.

This figure shows the movements of the chain yarns and weft yarns for a knitted structure from which a knit according to the invention can be obtained, having a chain sheet and two non-meshing sheets. Yarns A and A1 of the chain structure are shown in thick solid lines. Yarn A1 is a free chain yarn within the meaning of the present invention. The yarns of the first non-meshing sheet are shown in thin solid lines: these are the B yarns. The yarns of the second non-meshing sheet are shown in broken line: these are the C yarns.

In this example, the first guide bar, corresponding to the chain structure, is continuously full-threaded. The second guide bar, corresponding to the first non-meshing sheet and to the B yarns, is threaded continuously 1 full, 1 empty, 3 full, 1 empty, 1 full, 3 empty. The third guide bar, corresponding to the second non-meshing sheet and to the C yarns, is continuously threaded 1 full, 1 empty.

The knitting charts for these three sheets are as follows:
the chain sheet (yarns A and A1): 1-0/0-1//;
the first non-meshing sheet (B yarns): 1-1/3-3/2-2/0-0//;
the second non-meshing sheet (C yarns): 3-3/2-2/0-0/1-1//.

Thus, as FIG. 1 shows, no weft yarn passes all the way through chain yarn A1; in other words, each weft yarn, that is any B or C yarn, approaching said chain yarn A1, is optionally linked with said chain yarn A1, and then turns back one hundred and eighty degrees at this chain yarn A1.

The free chain yarn A1 can thus be unroved without affecting the adjacent chain yarns A, which are not free within the meaning of the present application, and therefore without destroying the structure of the knit on either side of this yarn A1. When this chain yarn A1 is pulled, the part of the knit lying on the left of this yarn A1 separates from the part lying on the right of this yarn A1 without unroving these two parts.

Moreover, because any weft yarn that approaches yarn A1 turns back one hundred and eighty degrees at this yarn A1, the edges of the separated parts have only one yarn B or C turning back through one hundred and eighty degrees and they are therefore smooth. No fraying occurs.

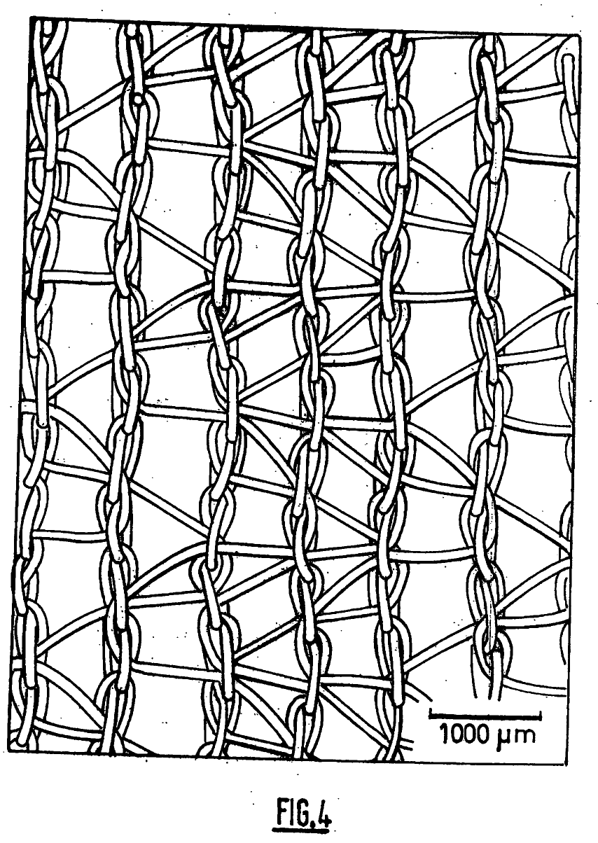

Such unroving of a free chain yarn A1 from a knitted structure from which a knit according to the invention can be obtained is visible in FIG. 3, which is the drawing of a photograph, taken with an Itashi S 800 scanning electron microscope, enlarge 20×, of an unroved part of such a knitted structure according to FIG. 1 and Example 1 of the present application. At the top of the figure, yarn A1 is unroved and the knitted parts on either side of this yarn A1 are intact. Their edges are smooth, only a C yarn does a one hundred and eighty degree turn. No yarn of the knit is cut or fraying. As appears clearly from FIG. 3, thanks to the specific threading of the yarns of the non-meshing sheets, the yarn C doing a one hundred and eighty degree turn at the edge of a separated part is integral with the core of said separated part, that is to say with the knit according to the invention. In this figure, yarn A1 is in the process of being unroved. Hence, at the bottom of the figure, yarn A1 is still knitted to the C yarns approaching it.

When unroving is complete, that is to say, when yarn A1 has been unroved all the way down the length of the knitted structure, yarn A1 is removed from said knitted structure and the two knitted parts lying on either side of this yarn A1 are completely separated. Repeating this unroving process on a second free chain yarn will completely separate a band from said knitted structure, this band being the knit according to the invention. The distribution of the A1 yarns predetermines with great precision the width of each tape.

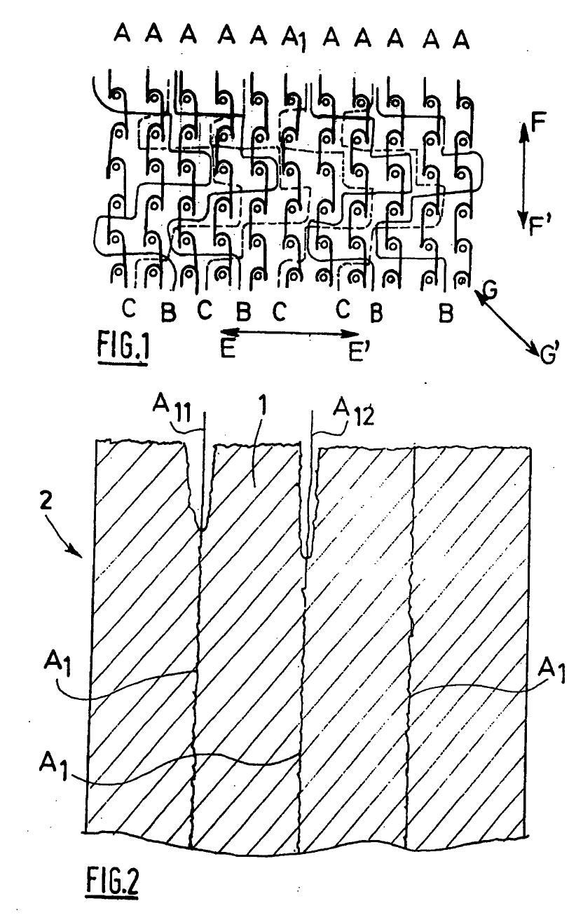

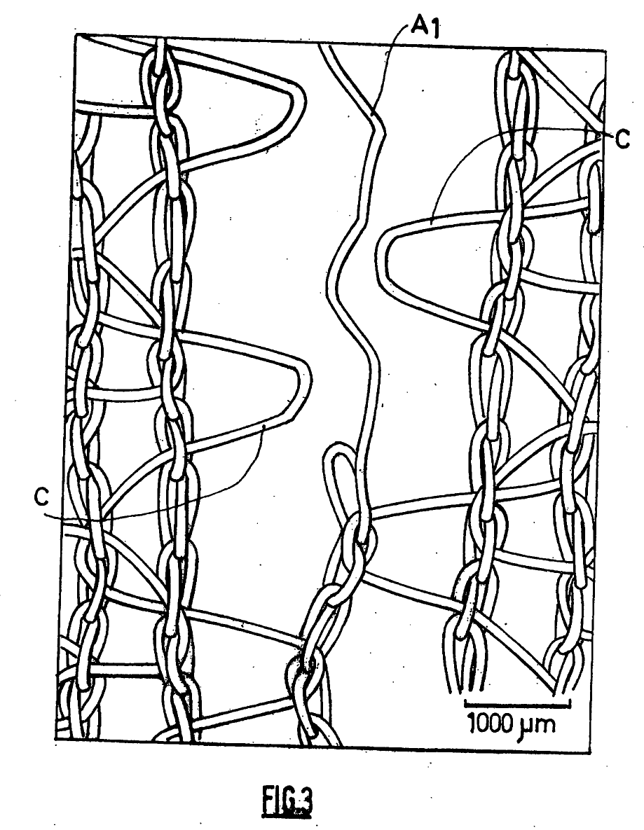

FIG. 2 shows diagrammatically a knitted structure 2 from which knits 1 according to the invention can be obtained. Two of the free chain yarns, yarns All and A12, are partially unroved. Complete unroving of these free chain yarns A11 and A12 thus produces a knit 1 according to the invention, that is a tape that can be used in the treatment of female stress urinary incontinence. Such tapes can also be used in the treatment of prolapse.

The center of a knit, tape or implant according to the invention is shown in FIG. 4, which is the drawing of a photograph taken under an Itashi S 800 scanning electron microscope, enlarged 20×. The knit or implant according to the invention corresponds to an area of said knitted structure 2 between two consecutive free chain yarns. The knit or implant does not therefore itself include any free chain yarns. There is therefore no risk of it unroving.

### EXAMPLE

A knitted structure from which a knit according to the invention can be obtained was made from 0.10 mm-diameter polypropylene monofilament yarn on a Raschel machine, with one chain sheet and two non-meshing sheets, in accordance with the following charts for the different sheets:
the chain sheet: 1-0/0-1//;
the first non-meshing sheet: 1-1/3-3/2-2/0-0//;
the second non-meshing sheet: 3-3/2-2/0-0/1-1// .

The first guide bar, corresponding to the chain structure, was continuously full-threaded. The second guide-bar, corresponding to the first non-meshing sheet, of partial weft, was continuously threaded 1 full, 1 empty, 3 full, 1 empty, 1 full, 3 empty. The third guide bar, corresponding to the second non-meshing sheet, of partial weft, was continuously threaded 1 full, 1 empty. The two partial wefts were threaded in such a way as to move under nine chain yarns, this making it possible eventually to obtain separate tapes, each about 1 cm wide. Thus, in this example, 1 chain yarn out of 10 was a free chain yarn within the meaning of the invention. The gage used was 24 needles.

The guide bars of the two non-meshing sheets moved in partial weft under three needles, symmetrically to each other, offset from each other in the direction of production of the knitted structure.

This knitted structure corresponds to the structure shown in FIG. 1 of the present application.

As it came off the machine, the knitted structure went through a heat-setting operation.

From this knitted structure, knits or tapes were produced by unroving at least two consecutive free chain yarns. The knits or tapes had the following characteristics:
thickness: approximately 0.3 mm;
diameter of cells: approximately 1 mm;
width: approximately 1 cm;
mass per unit area: approximately 50 g/m²;
tensile strength measured in accordance with method ISO 13934-1 on a tape 1 cm wide by 20 cm long: 66 N.

The knit or tape produced in this way by unroving at least two preferably consecutive free chain yarns from said knitted structure exhibits excellent tensile strength and is thus highly suitable for use as, or for the production of, a support implant for the treatment of stress urinary incontinence and prolapse.

For example, an implant having a length of 20 cm, or 30 cm or indeed 40 cm can be prepared from this tape. Such an implant has a very low mass per unit area. The amount of material implanted into the patient's body is therefore minimal.

Furthermore, owing to its method of manufacture, this implant has little elasticity and its edges, particular its longitudinal edges, are atraumatic, which means that it can be implanted without a protective sheath. Also, all knits, tape or implants obtained from the same knitted structure have uniform heat-setting and are easy to manipulate.

1. A prosthetic openwork knit for the treatment of urinary incontinence and/or prolapse, based on an arrangement of yarns of a biocompatible polymer comprising at least one first sheet defining a first chain structure, in which knit said arrangement of yarns further comprises at least two non-meshing sheets, of partial weft, the number of chain yarns in said chain structure being from 6 to 12.

2. The knit as claimed in claim 1, wherein the number of chain yarns is from 8 to 11, and is preferably 9.

3. The knit as claimed in claim 1, based on monofilament or multifilament yarns of a biocompatible polymer material selected from polypropylene, polyester, polyamide and blends thereof.

4. The knit as claimed in claim 3, in which said biocompatible polymer material is polypropylene.

5. The knit as claimed in claim 1, which is made from a blend of bioresorbable biocompatible yarns and non-bioresorbable biocompatible yarns.

6. The knit as claimed in claim 1, which is based on monofilament yarns having a diameter of from 0.05 mm to 0.15 mm, preferably approximately 0.10 mm.

7. The knit as claimed in claim 1, which has a thickness of from 0.20 mm to 0.40 mm, preferably approximately 0.30 mm.

8. The knit as claimed in claim 1, which comprises cells having a diameter of from 0.3 to 1.5 mm, preferably from 0.3 to 0.9 mm.

9. The knit as claimed in claim 1, which has a width of from 0.6 cm to 1.5 cm.

10. The knit as claimed in claim 1, the mass per unit area of which is from 40 to 75 g/m², and is preferably from 50 to 60 g/m².

11. The knit as claimed in claim 1, the tensile strength of which in the longitudinal and transverse directions, measured according to standard ISO 13934-1, is from 20 to 90 N, preferably from 40 to 90 N, preferably from 55 to 75 N, and more preferably from 60 to 70 N.

12. The knit as claimed in claim 1, which has an extension under 2 daN in the longitudinal direction, measured according to standard ISO 13934-1, of less than or equal to 15%, more preferably less than or equal to 10%.

13. The knit as claimed in claim 1, which comprises a first non-meshing sheet and a second non-meshing sheet, said first non-meshing sheet being in accordance with the chart 1-1/3-3/2-2/0-0//, said second non-meshing sheet being in accordance with the chart 3-3/2-2/0-0/1-1//.

14. The knit as claimed in claim 1, which is heat-set.

15. The knit as claimed in claim 1, which has a length of from 10 to 50 cm and constitutes a support implant for the treatment of stress urinary incontinence and/or prolapse.

16. The knit as claimed in, which has a number of stitch courses per centimeter ranging from 13 to 18, preferably of 15.

17. Use of a prosthetic knit as claimed in claim 1 to obtain a prosthetic product for surgical use, particularly to obtain a support implant for the treatment of stress urinary incontinence and/or prolapse.

18. A support implant for the treatment of stress urinary incontinence and/or prolapse, which is obtained by cutting transversely a prosthetic knit as claimed in claim 1.

19. The implant as claimed in claim 18, which has a length of from 10 to 50 cm.

20. A method of producing a prosthetic knit as claimed in claim 1, which comprises the following steps:
a) a knitted structure is produced on a warp or Raschel machine as a first sheet threaded continuously or as needed and obtained from a first guide bar, the chart followed for the knitting of the yarns of said first sheet leading to the formation of a chain, and at least a first non-meshing sheet and a second non-meshing sheet, said non-meshing sheets being threaded continuously or as needed, each said non-meshing sheet being obtained from a guide bar, the chart followed for the knitting of the yarns of each non-meshing sheet being such that for every n yams of the chain structure, n ranging from 6 to 12, every yarn of said non-meshing sheets that approaches chain yarn n+1 turns back one hundred and eighty degrees at said chain yarn n+1, said chain yarn n+1 being termed the free chain yarn, and
b) said free chain yarns are unroved along the length of the knitted structure obtained in step a) and said free chain yarns are removed to produce knits in which the number of chain yarns is from 6 to 12.

21. The method as claimed in claim 20, in which n is from 8 to 11.

22. The method as claimed in claim 21, in which n is 9.

23. The method as claimed in claim 22, in which the yarns of the first chain-structured sheet are knitted in accordance with a chart 1-0/0-1//, the yarns of the first non-meshing sheet are knitted in accordance with a chart 1-1/3-3/2-2/0-0//, and the yarns of the second non-meshing sheet are knitted in accordance with a chart 3-3/2-2/0-0/1-1//.

24. The method as claimed in claim 23, in which the guide bar of the first chain-structured sheet is continuously full-threaded, the guide bar of the first non-meshing sheet is continuously threaded 1 full, 1 empty, 3 full, 1 empty, 1 full, 3 empty, and the guide bar of the second non-meshing sheet is continuously threaded 1 full, 1 empty.

25. The method as claimed in claim 24, in which the two guide bars of the two non-meshing sheets move in partial weft under three needles, symmetrically with respect to each other, each offset with respect to the other in the direction of production of the knit.

26. The method as claimed in claim 20, in which the knitted structure is heat-set between step a) and step b).
